(19) United States
(12) Patent Application Publication
(10) Pub. No.: US 2008/0269495 A1
(43) Pub. Date: Oct. 30, 2008

(54) Process for Preparation of Piperidine Carboxylic Acid

(76) Inventors: Prosenjit Bose, Gurgaon (IN); Pramod Kumar, Hardoi (IN); Shilpi Mittal, Indore (IN); Yatendra Kumar, Gurgaon (IN)

Correspondence Address: RANBAXY INC., 600 COLLEGE ROAD EAST, SUITE 2100, PRINCETON, NJ, 08540, US

(21) Appl. No.: 11/573,071

(22) PCT Filed: Aug. 4, 2005
(86) PCT No.: PCT/IB05/52611

§ 371 Date: Aug. 1, 2007

(30) Foreign Application Priority Data
Aug. 4, 2004 (IN) 1448/DEL/2004

(51) Int. Cl. C07D 409/14

(52) U.S. Cl. 546/212

(57) ABSTRACT

A process for preparing pure tiagabine, which is a piperidine carboxylic acid compound, via pharmaceutically acceptable salts of tiagabine ester is provided. Also, L(+) tartaric acid, oxalic acid and dibenzoyl L(+) tartaric acid salts of tiagabine esters are provided. Further, process for preparing acid addition salts of tiagabine esters is provided.

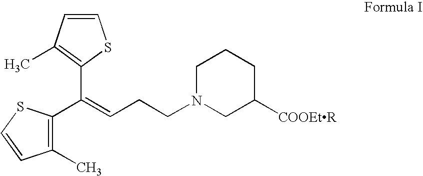

### TECHNICAL FIELD

Processes for preparing pure tiagabine, a piperidine carboxylic acid, using pharmaceutically acceptable acid addition salts of tiagabine esters are provided. L(+)-tartaric acid, oxalic acid and dibenzoyl L(+)-tartaric acid addition salts of tiagabine esters are also provided. Further, processes for preparing acid addition salts of tiagabine esters are provided.

### BACKGROUND OF THE INVENTION

Chemically, tiagabine is R(−)—N-(4,4-di(3-methylthien-2-yl)but-3-enyl)-nipecotic acid and is disclosed in U.S. Pat. No. 5,010,090. Tiagabine is an amino acid derivative exhibiting GABA (γ-aminobutyric acid, a neurotransmitter in the central nervous system)-uptake inhibitory properties and exerts useful pharmacological effects on the central nervous system by selectively enhancing the GABA activity.

U.S. Pat. No. 5,354,760 discloses a use of tiagabine ethyl ester hydrochloride for the preparation of crystalline tiagabine hydrochloride monohydrate. No other salt of tiagabine esters has been reported.

U.S. Pat. No. 5,010,090 also discloses the preparation of tiagabine from tiagabine ethyl ester, wherein tiagabine ethyl ester was purified by column chromatography on silica using methanol as eluent, which was then converted to tiagabine hydrochloride. Such a purification processes is cumbersome and expensive.

However, there remains a need for an improved process that avoids chromatographic techniques for preparing pure tiagabine. Such a process would be advantageous on a commercial scale.

### SUMMARY OF THE INVENTION

Provided herein are improved processes of preparing pure tiagabine and acid addition salts of tiagabine esters. In one aspect, provided are processes for preparing pure tiagabine comprising the steps of:
a) contacting crude tiagabine ester with one or more acids in one or more inert solvents to form an acid addition salt of tiagabine ester,
b) optionally isolating the acid addition salt of tiagabine ester as a solid, and
c) converting the acid addition salt of tiagabine ester into pure tiagabine or its pharmaceutically acceptable salts thereof.

Such processes can include one or more of the following embodiments. For example, the one or more inert solvents can be one or more alcohols, one or more esters, one or more ethers, one or more ketones, one or more nitriles, one or more chlorinated hydrocarbons, one or more cyclic ethers, one or more dipolar aprotic solvents or mixtures thereof. For example, alcohols can be methanol, ethanol, isopropanol, or mixtures thereof and ethers can be diethyl ether, diisopropyl ether, tertiary butyl methyl ether or mixtures thereof.

In another embodiment, acids can be one or more organic acids or one or more inorganic acids. Organic acids can be one or more of formic acid, acetic acid, succinic acid, maleic acid, malic acid, citric acid, ascorbic acid, mandelic acid, oxalic acid, tartaric acid, dibenzoyl tartaric acid, methanesulfonic acid, para toluenesulfonic acid, benzenesulfonic acid or mixtures thereof. In cases where chiral organic acids are used, dextro-rotatory isomers of such chiral acids can be used. Inorganic acids can be one or more of hydrochloric acid, hydrobromic acid, sulfuric acid, phosphoric acid, nitric acid or mixtures thereof.

In another embodiment, salts of tiagabine ester can be converted to pure tiagabine by acid hydrolysis or alkaline hydrolysis.

In yet another embodiment, pure tiagabine can be converted to its pharmaceutically acceptable acid addition salts. For example, pure tiagabine can be converted to tiagabine hydrochloride by contacting pure tiagabine with hydrochloric acid or hydrogen chloride gas.

In another embodiment, purification of tiagabine results in chiral purification or chemical purification. For example, chiral purity of the pure tiagabine or its pharmaceutically acceptable acid addition salts can be greater than about 99%, and in other embodiments, greater than about 99.5%. Chemical purity of the pure tiagabine or its pharmaceutically acceptable acid addition salts can be greater than about 98.5% by HPLC.

In another aspect, provided herein are acid addition salts of tiagabine ester of Formula I,

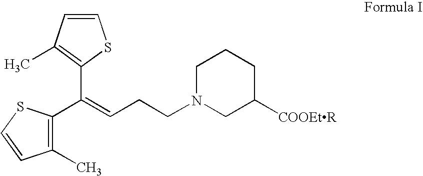

wherein R is L(+)-tartaric acid, oxalic acid or dibenzoyl L(+)-tartaric acid.

In yet another aspect, also provided are processes for preparing acid addition salts of tiagabine ester of Formula I,

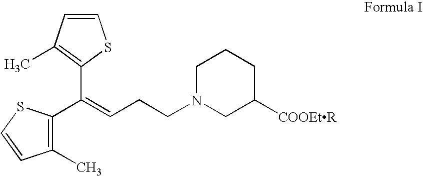

comprising contacting crude tiagabine ester with one or more acids in one or more inert solvents and isolating a corresponding acid addition salt of tiagabine ester.

Such processes can include one or more of the following embodiments. For example, acids can be one or more organic acids or one or more inorganic acids. Organic acids can be one or more of formic acid, acetic acid, succinic acid, maleic acid, malic acid, citric acid, ascorbic acid, mandelic acid, oxalic acid, tartaric acid, dibenzoyl tartaric acid, methanesulfonic acid, para toluenesulfonic acid, benzenesulfonic acid or mixtures thereof. In cases where chiral organic acids are used, dextro-rotatory isomers of such chiral acids can be used. Inorganic acids can be one or more of hydrochloric acid, hydrobromic acid, sulfuric acid, phosphoric acid, nitric acid or mixtures thereof.

In another embodiment, salts of tiagabine ester can be converted to pure tiagabine by acid hydrolysis or alkaline hydrolysis.

In another embodiment, pure tiagabine can be converted to its pharmaceutically acceptable acid addition salts.

In another embodiment, purification of tiagabine results in chiral purification or chemical purification. For example, chiral purity of the pure tiagabine or its pharmaceutically acceptable acid addition salts can be greater than about 99%, and in other embodiments, greater than about 99.5%. Chemical purity of the pure tiagabine or its pharmaceutically acceptable acid addition salts can be greater than about 98.5% by HPLC.

### DESCRIPTION OF THE DRAWINGS

FIG. 1 shows a powder X-ray diffraction pattern of L(+)-tartaric acid salt of tiagabine ethyl ester.

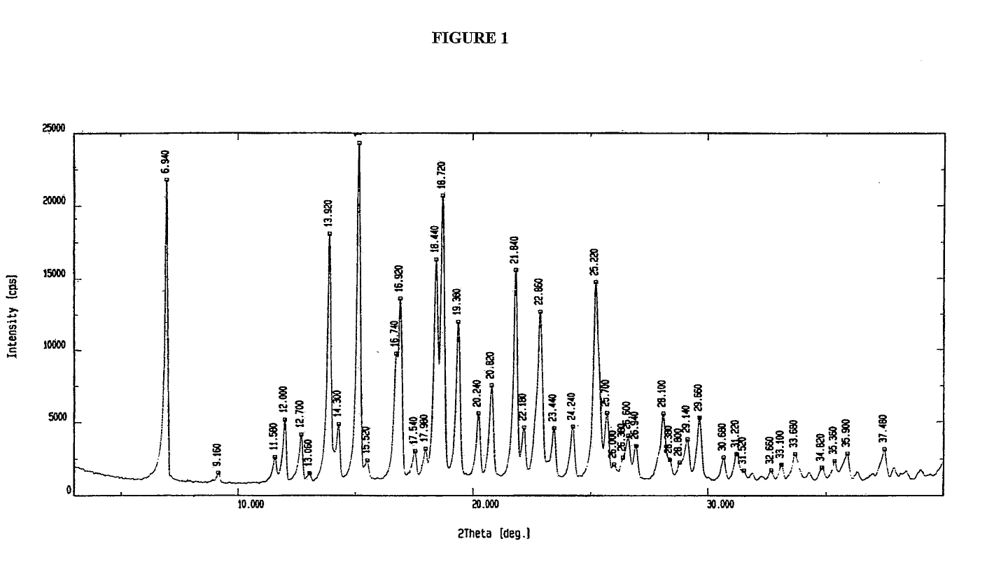

FIG. 2 shows powder X-ray diffraction pattern of oxalic acid salt of tiagabine ethyl ester.

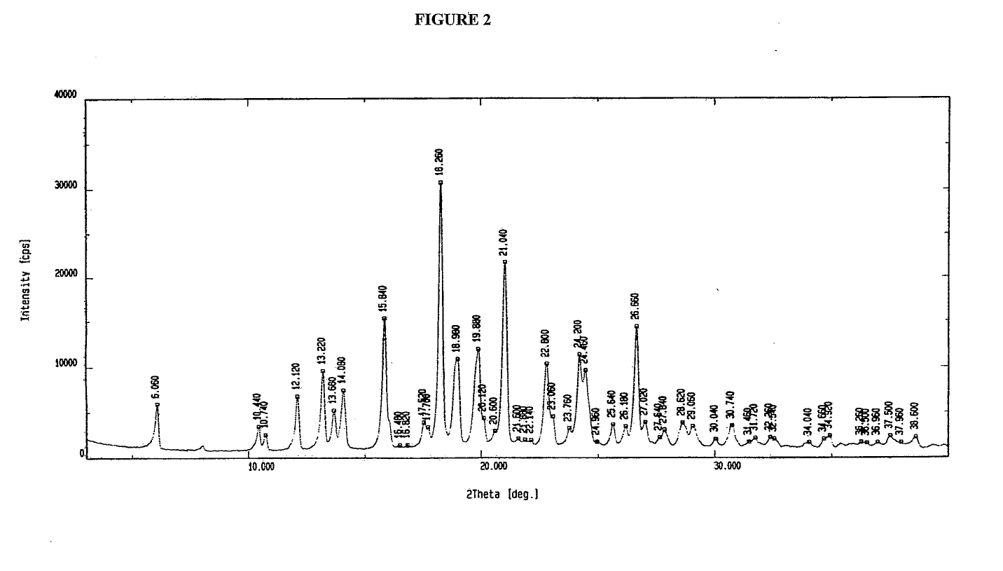

FIG. 3 shows powder X-ray diffraction pattern of dibenzoyl L(+)-tartaric acid salt of tiagabine ethyl ester.

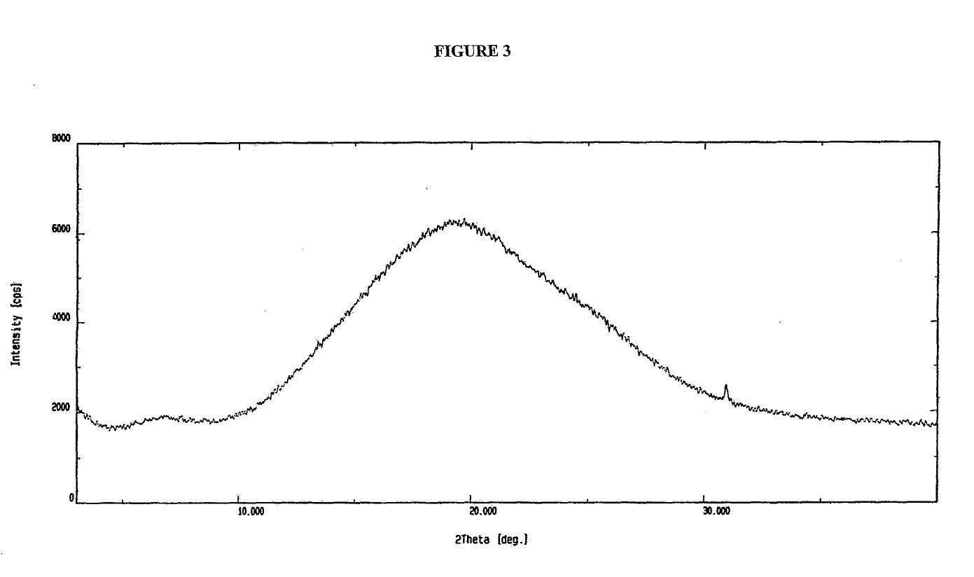

FIG. 4 shows an infrared absorption spectrum of L(+)-tartaric acid salt of tiagabine ethyl ester.

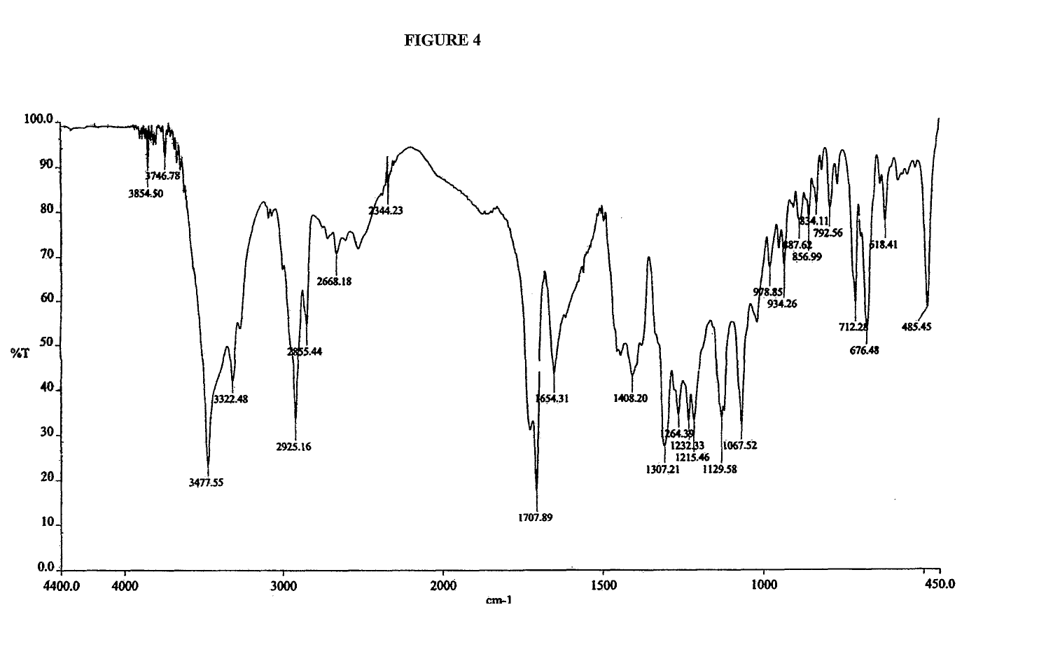

FIG. 5 shows an infrared absorption spectrum of oxalic acid salt of tiagabine ethyl ester.

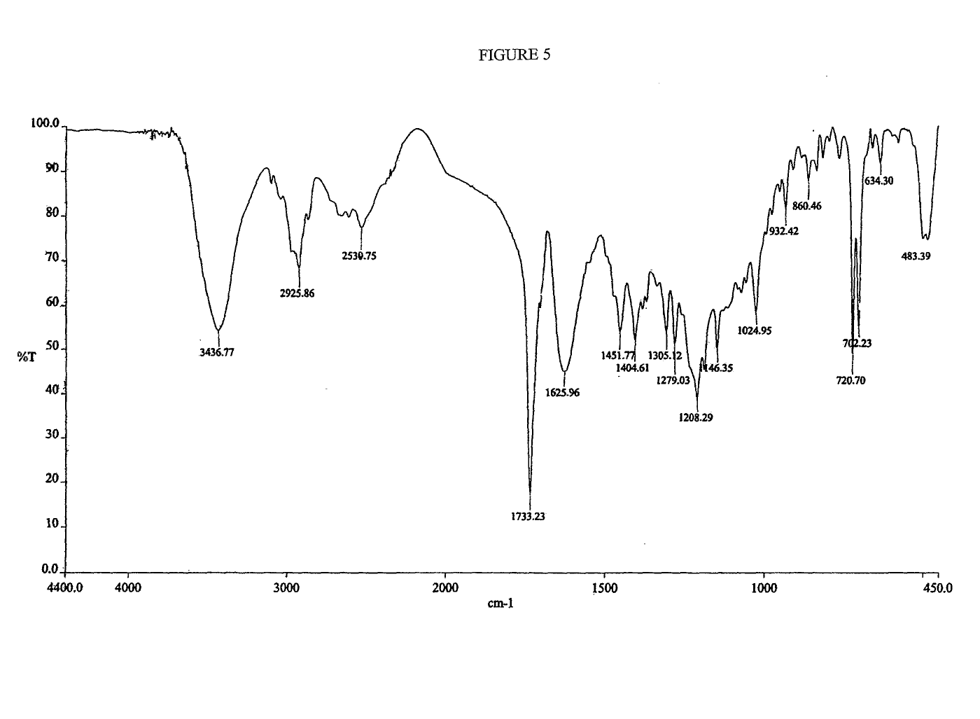

FIG. 6 shows an infrared absorption spectrum of dibenzoyl L(+)-tartaric acid salt of tiagabine ethyl ester.

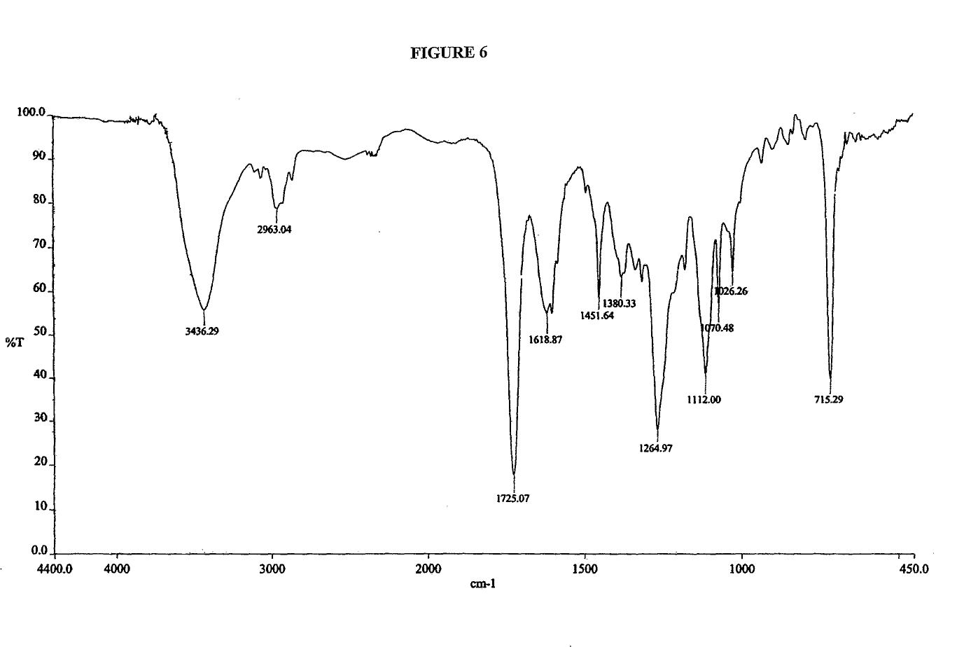

FIG. 7 shows DSC graph of L(+)-tartaric acid salt of tiagabine ethyl ester.

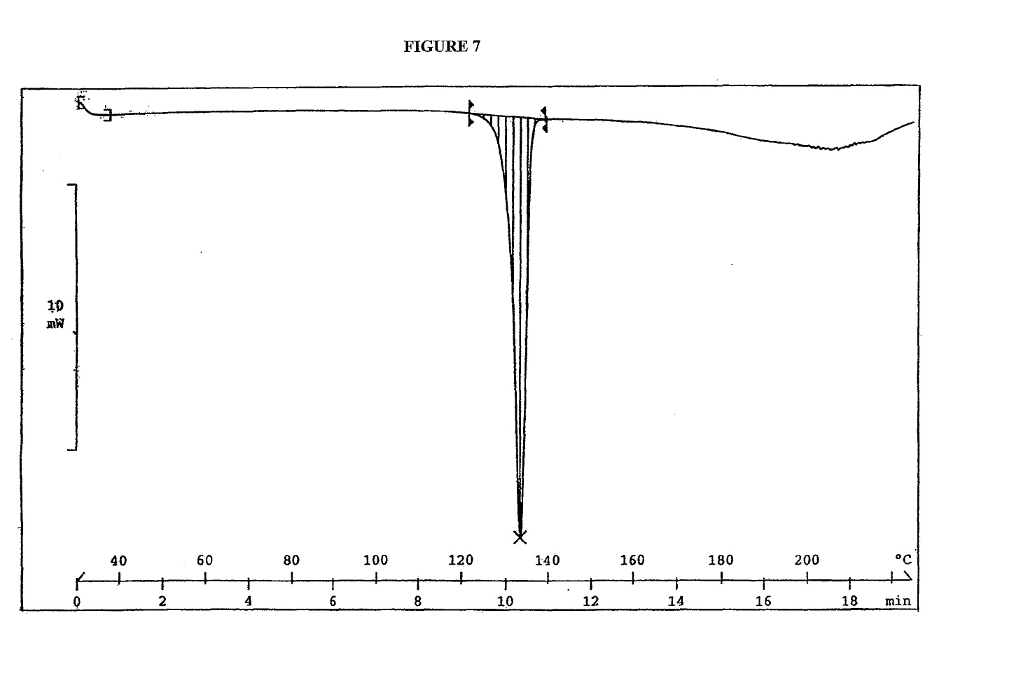

FIG. 8 shows DSC graph of oxalic acid salt of tiagabine ethyl ester.

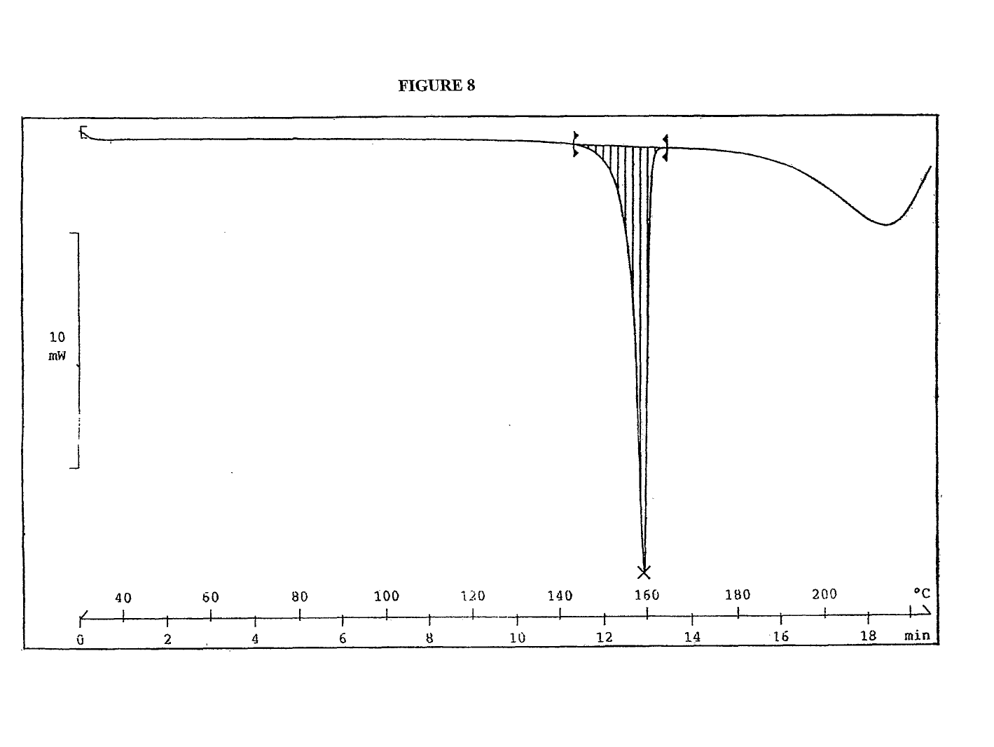

FIG. 9 shows DSC graph of dibenzoyl L(+)-tartaric acid salt of tiagabine ethyl ester.

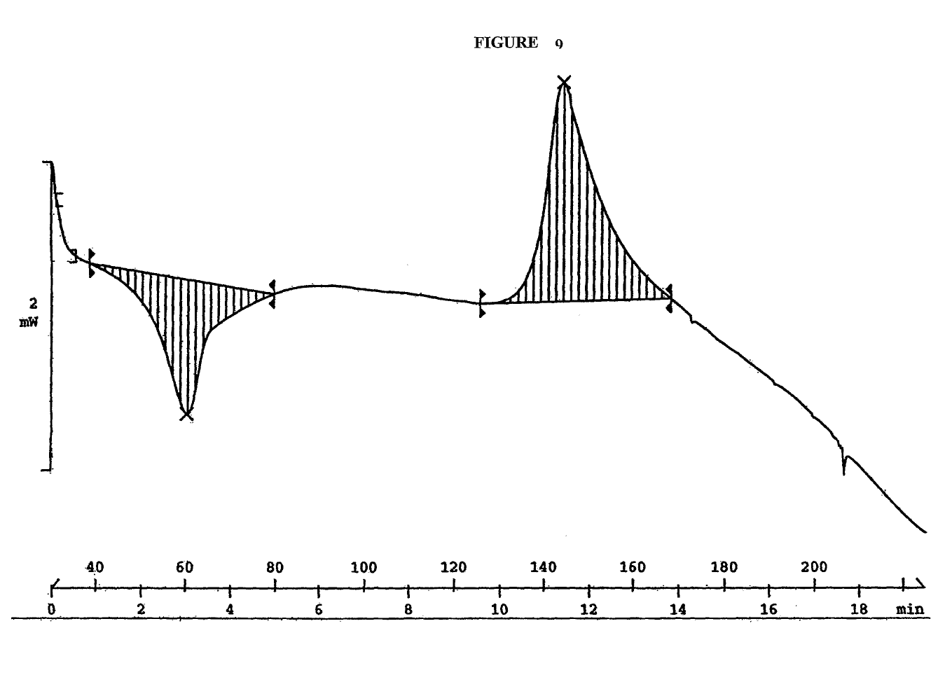

### DETAILED DESCRIPTION OF THE INVENTION

In one aspect provided are processes for preparing pure tiagabine comprising the steps of:
a) contacting crude tiagabine ester with one or more acid in one or more inert solvents to form an acid addition salt of tiagabine ester,
b) optionally isolating the acid addition salt of tiagabine ester in solid state, and
c) converting the acid addition salt of tiagabine ester into pure tiagabine or pharmaceutically acceptable salts thereof.

In another aspect, provided are organic acid addition salts of Formula I,

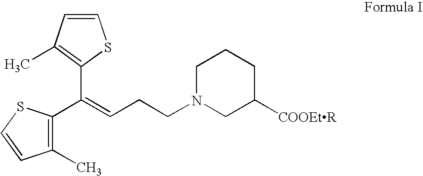

wherein R is L(+)-tartaric acid, oxalic acid or dibenzoyl L(+)-tartaric acid.

In yet another aspect, provided are processes for preparing acid addition salts of tiagabine ester comprising contacting tiagabine ester with one or more acids in one or more inert solvents and isolating the corresponding acid addition salts of tiagabine ester.

Crude tiagabine ester can be obtained by methods known in the art, for example, by a process disclosed in U.S. Pat. No. 5,010,090, which is incorporated herein in its entirety. Crude tiagabine ester can be utilized in the described processes as a solid or in solution form. For example, a solution of tiagabine ester may be obtained directly from the last step of a reaction in which tiagabine ester is formed and used for the preparation of acid addition salt of tiagabine ester.

The term “contacting,” as used herein, refers to mixing, dissolving, slurring, stirring or a combination thereof.

Examples of inert solvents utilized in the described processes include one or more alcohols (e.g., methanol, ethanol, isopropanol or mixtures thereof); ethers (e.g., diethyl ether, diisopropyl ether, tertiary butyl methyl ether or mixtures thereof); ketones (e.g., acetone, butanone or mixtures thereof); esters (e.g., ethylacetate, isopropylacetate or mixtures thereof); nitriles (e.g., acetonitrile); chlorinated hydrocarbons (e.g., methylene chloride, ethylenedichloride or mixtures thereof); dipolar aprotic solvents (e.g., dimethylsulfoxide, dimethylformamide or mixtures thereof); cyclic ethers (e.g., dioxane, tetrahydrofuran or mixtures thereof); or mixtures thereof.

Acid addition salts of tiagabine ester include, for example, salts with inorganic acids or organic acids. Examples of inorganic acids include, but are not limited to, hydrochloric acid, hydrobromic acid, sulfuric acid, phosphoric acid, or nitric acid. Examples of organic acids include, but are not limited to, formic acid, acetic acid, succinic acid, maleic acid, malic acid, citric acid, ascorbic acid, mandelic acid, oxalic acid, tartaric acid, dibenzoyl tartaric acid, methanesulfonic acid, para toluenesulfonic acid, or benzenesulfonic acid. Dextro-rotatory isomers of the above-described acids may be used for preparing chiral acid addition salts of tiagabine ester.

The described processes may be carried out at room or ambient temperatures, as well as higher temperatures for suitable durations required for the formation of the salt.

Salts of tiagabine ester may be isolated by concentration, crystallization, precipitation, cooling, filtration, centrifugation or combinations thereof. Precipitation of salts of tiagabine ester may be spontaneous, depending upon solvents used and reaction conditions. Precipitation may also occur upon addition of one or more antisolvents, i.e., solvents in which salt of tiagabine ester is insoluble or sparingly soluble, to the inert solvent(s) in which salts of tiagabine ester are prepared. Alternatively, precipitation may be induced by concentration and/or reducing the temperature of the inert(s) solvent, particularly if the initial temperatures are elevated.

Examples of antisolvents that may be added to precipitate out salts of tiagabine ester include, but are not limited to, hydrocarbons (e.g., hexane, cyclohexane, toluene, heptane, octane or mixtures thereof); lower alkyl ethers (diethylether, diisopropylether or. mixtures thereof); or mixtures thereof.

Times and temperatures for crystallizations/precipitations are not critical. For example, the crystallization/precipitation may be performed at temperatures from about 5° C. to about 40° C. and for times of about 30 minutes to about 3 hours in some embodiments.

Salts of tiagabine ester in solid state can be isolated to assist in the removal of impurities. For example, salts of tiagabine ester may be crystallized one or more times before conversion to tiagabine to provide higher purity tiagabine. In another example, salts of tiagabine ester in crystalline form can be isolated to assist in obtaining higher purity tiagabine.

Solvent amounts may be varied depending on the type of solvent(s), lot size etc. Operation conditions, for example stirring, are not limited for the described processes, and in some embodiments, crystallization or precipitation may be conducted with or without stirring.

Conversion of salts of tiagabine ester to pure tiagabine may be achieved by acid hydrolysis, alkali hydrolysis or hydrogenation, particularly, for example, when benzyl esters are used. Hydrogenations may be carried out by using convention methods known to one of ordinary skill in the art, and in particular, can be carried out in the presence of one or more metal catalysts. Metal catalysts that may be used in hydrogenations include palladium, nickel and platinum. Acid hydrolyses and base hydrolyses may be carried out using procedures well known to one of ordinary skill in the art. For example, reagents for acid hydrolyses include one or more mineral acids, for example, haloacids (HCl, HBr, and the like or mixtures thereof), sulfuric acid and other mineral acids; and reagents for base hydrolyses include various mineral hydroxides, for example, Group I hydroxides (e.g., sodium hydroxide, potassium hydroxide, and the like, or mixtures thereof).

Solvents used to convert salts of tiagabine ester to pure tiagabine or its pharmaceutically acceptable salts are not critical and may be the same as those used for the preparation of salts of tiagabine ester as described above. For example, solvents that may be used in this conversion step include one or more alcohols (e.g., methanol, ethanol, isopropanol or mixtures thereof); ethers (e.g., diethyl ether, diisopropyl ether, tertiary butyl methyl ether or mixtures thereof); ketones (e.g., acetone, butanone or mixtures thereof); esters (e.g., ethylacetate, isopropylacetate or mixtures thereof); nitrites (e.g., acetonitrile); chlorinated hydrocarbons (e.g., methylene chloride, ethylenedichloride or mixtures thereof); dipolar aprotic solvents (e.g., dimethylsulfoxide, dimethylformamide or mixtures thereof); cyclic ethers (e.g., dioxane, tetrahydrofuran or mixtures thereof); or mixtures thereof.

Reaction times and temperatures are not critical. For example, the reaction may be performed at temperatures from about 20° C. to about 80° C. and at reaction times from about 1 hour to about 6 hours in some particular embodiments.

Pure tiagabine may be isolated in a manner similar to that detailed above for isolating salt of tiagabine ester. For example, pure tiagabine may be isolated by concentration, crystallization, precipitation, cooling, filtration, centrifugation or combinations thereof.

Tiagabine may be converted to its pharmaceutically acceptable acid addition salts by adding the corresponding acid in one or more suitable solvents. For example, tiagabine hydrochloride may be prepared by contacting tiagabine with HCl (e.g., hydrogen chloride gas or hydrochloric acid).

Isolation of acid addition salts of tiagabine ester as intermediates in processes for preparing pure tiagabine or its pharmaceutically acceptable salts results in chemical purification, as well as chiral purification. Tiagabine or its pharmaceutically acceptable salts of chemical purity of more than about 99% may be obtained by the described processes. Chemical purities of tiagabine or its pharmaceutically acceptable salts may be more than 98.5% in some embodiments. Tiagabine or its pharmaceutically acceptable salts of chiral purity of more than about 99.5% may also be obtained by the described processes. Chiral purities of tiagabine or its pharmaceutically acceptable salts may be more than 99.9% in some embodiments.

Pure tiagabine or its pharmaceutically acceptable salts thereof having less than about 0.5% of impurities at RRT 1.13 (as per USP monograph USP 26-NF 21 suppl.) can be obtained by the present process. Pure tiagabine or its pharmaceutically acceptable salts thereof having less than about 0.3% impurities, and even less than about 0.1% impurities may be obtained in some embodiments.

L(+)-tartaric acid salt (i.e., L(+)-tartarate salt) of tiagabine ethyl ester may be obtained as a crystalline material. Such L(+)-tartaric acid salt of tiagabine ethyl ester may be characterized by XRD spectra having X-ray peaks at about 6.94, 13.92, 15.18, 16.92, 18.44, 18.72, 19.38, 21.84, 22.86 and 25.22±0.2 degrees two-theta. Oxalic acid salt of tiagabine ethyl ester may be obtained as a crystalline material. Such oxalic acid salt of tiagabine ethyl ester may be characterized by XRD spectra having strong X-ray peaks at about 15.84, 18.26, 21.04 and 26.66±0.2 degrees two-theta and weak peaks at about 13.22, 18.98, 19.88, 24.20 and 24.46±0.2 degrees two-theta. Dibenzoyl L(+)-tartaric acid salt of tiagabine ethyl ester may be obtained in an amorphous form. Salts described herein may also be characterized by their IR and DSC graphs.

While the present invention has been described in terms of its specific embodiments, certain modifications and equivalents will be apparent to those skilled in the art and are included within the scope of the present invention. The examples are provided to illustrate particular aspects of the disclosure and do not limit the scope of the present invention as defined by the claims.

### EXAMPLES

### Methods

### Powder XRD:

### X-Ray Difractometer, Rigaku Coorperation, RU-H3R Model Dmax 2500H

X-Ray tube with Cu target anode
Divergence slits 1°, Receiving slit 0.15 mm, Scatter slit 1°

### Power: 40 KV, 100 mA

Scanning speed: 2 deg/min step: 0.02 deg
Wave length: 1.5406 A

### FT-IR:

### Instrument: Perkin Elmer, Spectrum One

SCAN: 16 scans, 4.0 cm⁻¹
according to the USP 25, general test methods page 1920, infrared absorption spectrum by potassium bromide pellet method.

### DSC:

### DSC821 e, Mettler Toledo

Sample weight: 2-5 mg
Temperature range: 30-225° C.
Heating rate: 10° C./min
Nitrogen 50.0 mL/min
Number of holes in the crucible: No hole

### HPLC:

### HPLC-Column LUNA C-18 (150×4.6) 5μ

### Example 1

### Preparation of Tiagabine Ethyl Ester-L (+) Tartrate Salt

L (+) tartaric acid (3.72 g) was added to a stirred solution of crude tiagabine ethyl ester (14.2 g, HPLC Purity=70%) in isopropanol (100 mL) at ambient temperature. The mixture was stirred at about 70-80° C. for about 1 hour resulting in a clear solution. The hot solution was filtered to remove insoluble material and the filtrate cooled and stirred at room temperature for 4 hours to crystallize the product. The obtained product was recrystallized from isopropanol to yield pure title compound.

HPLC Purity: 99.13%

Melting Point: 129-130° C.

Yield: 9.7 g

### Example 2

### Preparation of Tiagabine Ethyl Ester-Oxalate Salt

A solution of oxalic acid (3.0 g) in isopropanol was added to a stirred solution of crude tiagabine ethyl ester (12.0 g, HPLC purity=80%) in isopropanol at ambient temperature. The mixture was stirred at about 70-80° C. for about 2 hours resulting in a clear solution.

The hot solution was allowed to cool to room temperature and was stirred for about 4 hours to crystallize the product. The obtained product was filtered and recrystallized from isopropanol to yield pure title compound.

HPLC Purity: 98.74%

Melting Point: 154-155° C.

Yield: 8.8 g

### Example 3

### Preparation of Tiagabine Ethyl Ester-Dibenzoyl L (+) Tartrate Salt

A solution of dibenzoyl L (+) tartaric acid (0.85 g) in isopropyl ether was added to a stirred solution of crude tiagabine ethyl ester (1.2 g, HPLC purity=80) in isopropyl ether (20 mL) at ambient temperature. The mixture was stirred for about 2 hours at room temperature to crystallize the product. The obtained product was filtered and recrystallized from isopropyl ether to yield pure title compound.

HPLC Purity: 99.08%

Melting Point: 70-72° C.

Yield: 1.6 g

### Example 4

### Preparation of Tiagabine Hydrochloride from Tiagabine Ethyl Ester-L (+) Tartrate Salt

A solution sodium hydroxide (10.8 ml, 8M) was added to a stirred solution of L (+) tartaric acid salt of tiagabine ethyl ester (12 g, purity: >99.5%) in ethanol at ambient temperature. The solution was stirred for about 3 to 5 hours until completion of the reaction. The mixture was diluted with water (50 mL) and acidified with dilute hydrochloric acid until a pH of about 1.0 was obtained. The acidic solution was extracted twice with ethyl acetate (100 mL). The ethyl acetate layer was then washed with water (25 mL) and concentrated by evaporation under vacuum to yield crude product. Crude tiagabine hydrochloride was recrystallized from ethanol to yield pure tiagabine hydrochloride.

HPLC Chiral Purity: 99.9%

HPLC purity: 99.9%

Yield: 6.3 g

Impurity at RRT 1.13: 0.07%

(By HPLC)

We claim:

1. A process for preparing pure tiagabine comprising the steps of:
a) contacting crude tiagabine ester with one or more acids in one or more inert solvents to form an acid addition salt of tiagabine ester,
b) optionally isolating the acid addition salt of tiagabine ester as a solid, and
c) converting the acid addition salt of tiagabine ester into pure tiagabine or its pharmaceutically acceptable salts thereof.

2. The process of claim 1, wherein the one or more inert solvents are selected from one or more alcohols, one or more esters, one or more ethers, one or more ketones, one or more nitriles, one or more chlorinated hydrocarbons, one or more cyclic ethers, one or more dipolar aprotic solvents or mixtures thereof.

3. The process of claim 2, wherein the one or more alcohols are selected from methanol, ethanol, isopropanol, or mixtures thereof and the one or more ethers are selected from diethyl ether, diisopropyl ether, tertiary butyl methyl ether or mixtures thereof.

4. The process of claim 1, wherein the one or more acids are selected from one or more organic acids or one or more inorganic acids.

5. The process of claim 4, wherein the one or more organic acids are selected from formic acid, acetic acid, succinic acid, maleic acid, malic acid, citric acid, ascorbic acid, mandelic acid, oxalic acid, tartaric acid, dibenzoyl tartaric acid, methanesulfonic acid, para toluenesulfonic acid, benzenesulfonic acid or mixtures thereof.

6. The process of claim 5, wherein the one or more organic acids are chiral and dextro-rotatory isomers of the chiral acids are used.

7. The process of claim 4, wherein the one or more inorganic acids are selected from hydrochloric acid, hydrobromic acid, sulfuric acid, phosphoric acid, nitric acid or mixtures thereof.

8. The process of claim 1, wherein salts of tiagabine ester are converted to pure tiagabine by acid hydrolysis or alkaline hydrolysis.

9. The process of claim 8, wherein the pure tiagabine is converted to its pharmaceutically acceptable acid addition salts.

10. The process of claim 9, wherein the pure tiagabine is converted to tiagabine hydrochloride by contacting pure tiagabine with hydrochloric acid or hydrogen chloride gas.

11. The process of claim 1, wherein chiral purity of the pure tiagabine or its pharmaceutically acceptable acid addition salts is greater than about 99%.

12. The process of claim 1, wherein chiral purity of the pure tiagabine or its pharmaceutically acceptable acid addition salts is greater than about 99.5%.

13. The process of claim 1, wherein chemical purity of the pure tiagabine or its pharmaceutically acceptable acid addition salts is greater than about 98.5% by HPLC.

14. An acid addition salt of tiagabine ester of Formula I, 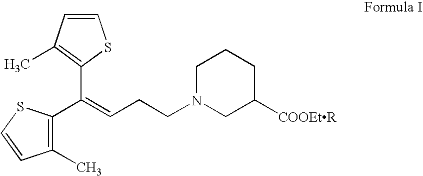
wherein R is L(+)-tartaric acid, oxalic acid or dibenzoyl L(+)-tartaric acid.

15. A process for preparing acid addition salts of tiagabine ester of Formula I, 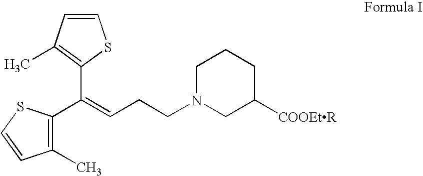
comprising contacting crude tiagabine ester with one or more acids in one or more inert solvents and isolating a corresponding acid addition salt of tiagabine ester.

16. The process of claim 15, wherein the one or more acids are selected from one or more organic acids or one or more inorganic acids.

17. The process of claim 16, wherein the one or more organic acids are selected from formic acid, acetic acid, succinic acid, maleic acid, malic acid, citric acid, ascorbic acid, mandelic acid, oxalic acid, tartaric acid, dibenzoyl tartaric acid, methanesulfonic acid, para toluenesulfonic acid, benzenesulfonic acid or mixtures thereof; or the one or more inorganic acids are selected from hydrochloric acid, hydrobromic acid, sulfuric acid, phosphoric acid, nitric acid or mixtures thereof.

18. The process of claim 16, wherein the one or more organic acids are chiral and dextro-rotatory isomers of the chiral acids are used.

19. The process of claim 15, wherein the salts of tiagabine ester is converted to pure tiagabine by acid hydrolysis or alkaline hydrolysis.

20. The process of claim 15, wherein the pure tiagabine is converted to its pharmaceutically acceptable acid addition salts.

21. The process of claim 15, wherein chiral purity of the pure tiagabine or its pharmaceutically acceptable acid addition salts is greater than about 99%; or chemical purity of the pure tiagabine or its pharmaceutically acceptable acid addition salts is greater than about 98.5% by HPLC.
